(19) United States
(12) Patent Application Publication
(10) Pub. No.: US 2007/0086564 A1
(43) Pub. Date: Apr. 19, 2007

(54) Method for calibrating a CT system having at least two focus/detector systems arranged angularly offset from one another, and computed tomography system

(76) Inventors: Herbert Bruder, Hochstadt (DE); Martin Petersilka, Adelsdorf (DE)

Correspondence Address: HARNESS, DICKEY & PIERCE, P.L.C., P.O.BOX 8910, RESTON, VA, 20195, US

(21) Appl. No.: 11/543,931

(22) Filed: Oct. 6, 2006

(30) Foreign Application Priority Data
Oct. 12, 2005 (DE) 10 2005 048 891.9

(51) Int. Cl. A61B 6/00; G01N 23/00; G21K 1/12; H05G 1/60

(52) U.S. Cl. 378/9

(57) ABSTRACT

A method is disclosed for calibrating a CT system having at least two focus/detector systems which are fastened on a rotatable gantry and are arranged angularly offset from one another, in order to scan a patient the angularly offset foci with fanned-open X-ray beams irradiating the respectively oppositely situated detectors with a multiplicity of detector elements arranged like matrices, while the focus/detector systems rotate about the object, preferably a patient, moved, if appropriate, along a system axis, and each detector element of each focus/detector system is assigned an X-ray beam per angle of rotation of the gantry. According to an embodiment of the method, the measured values of the at least two focus/detector systems are coordinated with one another individually per measured X-ray beam before the carrying out of a reconstruction of CT data of the object or patient from at least two different focus/detector systems by means of a calibration matrix (K_{k,s,r}^{FDSA}, K_{k,s,r}^{FDSB}) per focus/detector system, each calibration matrix (K_{k,s,r}^{FDSA}, K_{k,s,r}^{FDSB}) being determined in such a way that it generates a compensation between measured values during simultaneous operation of the at least two focus/detector systems, on the one hand, and absorption data mutually uninfluenced by the number of focus/detector systems, on the other hand.

### PRIORITY STATEMENT

The present application hereby claims priority under 35 U.S.C. §119 on German patent application number DE 10 2005 048 891.9 filed Oct. 12, 2005, the entire contents of which is hereby incorporated herein by reference.

### FIELD

The invention generally relates to a method for calibrating a CT system. For example, it may relate to one in which at least two focus/detector systems, arranged angularly offset from one another, are arranged on a rotatable gantry, in order to scan an object, preferably a patient. Further, it may relate to one in which the angularly offset foci with fanned-open X-ray beams irradiate the respectively oppositely situated detectors with a multiplicity of detector elements arranged like matrices, while the focus/detector systems rotate about the object, and each detector element of each focus/detector system is assigned an X-ray beam per angle of rotation of the gantry. The invention further generally relates to a computed tomography system.

### BACKGROUND

The laid-open patent application DE 10302565 A1 discloses a tomography unit having two focus/detector systems that are arranged in an angularly offset fashion and with the aid of which an object, preferably a patient, can be scanned by rotating the focus/detector systems. By contrast with a simple CT having one focus/detector system, it is possible to use such a CT having a number of focus/detector systems to achieve a higher temporal resolution. This is helpful, in particular, when recording cyclically moving objects such as the heart, for example.

Such detector systems are calibrated before being used, as is also known in the case of CT systems with simple focus/detector systems. It is generally the case here that an air calibration, a normalization to a dose monitor value, a radiation hardening correction, a channel correction and a water scaling are performed.

However, it has emerged from the operation of such CT systems having at least two focus/detector systems arranged angularly offset from one another that artifacts occur that are to be ascribed to a lack of calibration between the focus/detector systems.

In the German Patent Application 10 2004 062 857.2 from the applicant, which is not a prior publication, it is proposed to undertake in the case of a CT system having a number of focus/detector systems scaling between the focus/detector systems that can, if appropriate, also be prepared individually for the individual channels of a projection, if appropriate for different projection angles.

However, it has emerged that this type of calibration does not suffice to eliminate the artifacts that are produced when measured values of the different focus/detector systems are mixed for the reconstruction.

A CT system having a number of focus/detector systems is known from US 6 876 719 B2. Correction values with different combinations of energized X-ray sources are determined from measurements at a patient in order to correct scattered radiation artifacts. The scattered radiation artifacts in recorded projection data are corrected on the basis of the determined correction values.

### SUMMARY

At least one embodiment of the invention represents an improved calibration method and/or computed tomography system that enable a further suppression of artifacts and, as far as possible, an elimination of existing artifacts in CT systems having a number of focus/detector systems.

The inventors, in at least one embodiment, have realized that it is more favorable to calibrate the individual focus/detector systems to uninfluenced measured values instead of merely mutually fitting the measuring systems. By way of example, measurements that have come about exclusively with the aid of a single focus/detector system can be used as uninfluenced measuring systems. On the other hand, however, it is also possible to carry out an ideal measurement by means of an analytical calculation of absorption data on the basis of known phantom absorption values and to coordinate the calibration thereon.

Consequently, the inventors, in at least one embodiment, propose a method for calibrating a CT system, in which at least two focus/detector systems arranged angularly offset from one another are arranged on a rotatable gantry, in order to scan an object, preferably a patient, the angularly offset foci with fanned-open X-ray beams irradiate the respectively oppositely situated detectors with a multiplicity of detector elements arranged like matrices, while the focus/detector systems rotate about the object, and each detector element of each focus/detector system is assigned an X-ray beam per angle of rotation of the gantry, and the measured values of the at least two focus/detector systems are coordinated with one another individually per measured X-ray beam before the carrying out of a reconstruction of CT data of the object from at least two different focus/detector systems by way of a calibration matrix per focus/detector system, each calibration matrix being determined in such a way that it generates a compensation between measured values during simultaneous operation of the at least two focus/detector systems, on the one hand, and absorption data mutually uninfluenced by the number of focus/detector systems, on the other hand.

In order to determine the calibration matrix, it is possible in a particular variant design, in at least one angular position of the gantry, to carry out a scan of at least one phantom simultaneously with the aid of all the focus/detector systems and to calculate the theoretical attenuation of the X-ray beam at this at least one phantom for each measured X-ray beam of each focus/detector system, each calibration matrix subsequently being prepared on the basis of the calculated beams, such that each measured X-ray beam of each focus/detector system is normalized to the calculated attenuation of the corresponding X-ray beam.

It is possible on the basis of symmetry properties for the calculation of the attenuation values and the scanning of the phantom to take place at a single angle of rotation in the case of a rotationally symmetrical phantom, and for each calibration matrix to be prepared independently of the angle of rotation of the gantry. However, it is to be noted here that the influence of a patient couch, which is certainly slight but present nonetheless, remains out of account under these circumstances.

In another refinement of this method according to at least one embodiment of the invention, the calculation of the attenuation values and the scanning of the phantom can take place for a multiplicity of angles of rotation, and each calibration matrix can be prepared for all the spatial directions of the beams.

A fundamentally different variant for the analytical calculation of absorption values can consist in that a scan is carried out simultaneously with the aid of all the focus/detector systems in at least one angular position of the gantry of at least one phantom, a scan is carried out with the aid of only one focus/detector system, and the attenuation of the X-ray beams at this at least one phantom is determined without the influence of the at least one other focus/detector system, each calibration matrix being prepared on the basis of the attenuation values of the beams determined with the aid of only one focus/detector system, and each measured X-ray beam of each focus/detector system being normalized to the individually determined attenuation of the corresponding X-ray beam. Thus, possible mutual influence between the focus/detector systems is excluded in this case owing to the fact that the measured data of a focus/detector system operating alone is used as basis for forming the calibration matrix. Such a method is advantageous particularly when the structure of the scanned object on which the calibration is carried out is asymmetric, or can be represented computationally only with difficulty such that an analytical approach would be complicated.

The determination of the attenuation of the X-ray beams can be carried out here very easily by a single focus/detector system and the scan can be carried out for a multiplicity of angles of rotation with the aid of all the focus/detector systems, and each calibration matrix can be prepared for all the spatial directions of the beams.

In the case of the abovenamed embodiments, typical body shapes, for which calibration matrices are stored in each case, can be used as the phantom, calibration matrices being used for the most similar shape and dimension in each case in accordance with the scanned object region.

If the scanned object has different cross sections such as is the case, for example, with a patient, it is particularly advantageous when the adaptation and/or selection of each calibration matrix is performed by at least one topogram recorded before scanning the patient.

In order to determine an optimum calibration matrix or to adapt it to the dimensions and the shape of the scanned object, at least two topograms recorded in an angularly offset fashion before scanning can be used. The actual extents of the object in at least two planes can thereby be determined, and it is thus possible to make a relatively accurate selection of calibration matrices to be used in the respective scanning plane.

Again, the adaptation and/or selection of each calibration matrix can be performed on the basis of topograms recorded in an angularly offset fashion with the aid of each focus/detector system, the relative recording angles in relation to one another corresponding to the angular offset of the focus/detector systems on the gantry.

As an alternative to preparing topograms and to the adaptation and/or selection, being effected therefrom, of each calibration matrix, the extent of the scanned object can also be determined immediately during scanning with the aid of the object shadows measured in the process. For example, a threshold value can then be set to this end such that all the absorption values above this threshold value form the object shadow, and it is thereby possible to infer the extent of the scanned object.

According to an embodiment of the invention, the method described can be carried out by projection with reference to the calibration, it also being possible with particular advantage to carry out the calibration with the aid of parallel projection.

In a particular refinement of the method according to an embodiment of the invention, during the determination of the calibration matrices in the case of a two-focus/detector system the calibration matrix K_{k,s,r}^{FDSA} of the first focus/detector system FDSA, and the calibration matrix K_{k,s,r}^{FDSA} of the second focus/detector system FDSB can be calculated as follows according to the formulae:

$${K}_{k , s , r}^{FDSA} = 1 + \frac{{W}_{k , s , r} ( {x}_{0} , {y}_{0} , {α}_{0}^{FDSA} ) - {h}_{k , s , r}^{FDSA}}{{W}_{k , s , r} ( {x}_{0} , {y}_{0} , {α}_{0}^{FDSA} )}$$

$$and$$

$${K}_{k , s , r}^{FDSB} = 1 + \frac{{W}_{k , s , r} ( {x}_{0} , {y}_{0} , {α}_{0}^{FDSB} ) - {h}_{k , s , r}^{FDSB}}{{W}_{k , s , r} ( {x}_{0} , {y}_{0} , {α}_{0}^{FDSB} )}$$

where W_{k,s,r} (x₀, y₀, α₀^{FDSA}) and W_{k,s,r} (x₀, y₀, α₀^{FDS}) are the “true” projection values as calculated or measured in individual operation, h_{k,s,r}^{FDSA} are the measured data, obtained during a common scan, of the focus/detector system FDSA, and h_{k,s,r}^{FDSA} are the measured data of the FDSB focus/detector system k,s,r, k determining the channel of a projection, s determining the row of the detector, r determining the projection number, x₀, y₀ determining the position of the phantom, preferably of a water disk, and α₀^{FDSA} and α₀^{FDSB} respectively determining the projection angles of the respective focus/detector system.

If the method according to an embodiment of the invention is used for CT systems having detectors of different size or used ray fans of different size, the values of the smaller detector or ray fan should be calibrated to the values of the larger detector or ray fan.

When carrying out the method according to an embodiment of the invention, the object can be moved along a system axis during the rotation of the focus/detector systems.

In accordance with the method according to an embodiment of the invention outlined above and its embodiments, the inventors also propose a computed tomography system having at least two focus/detector systems that scan an object with the aid of different ray fans, the attenuation of the radiation during passage through the object being determined, and tomograms or volume data of the spatial attenuation of the object being determined therefrom with the aid of a computation unit and programs or program modules stored therein, program code for carrying out the previously described method and, if appropriate, also refinements thereof being contained in the programs or program modules.

### BRIEF DESCRIPTION OF THE DRAWINGS

The invention is described in more detail below using an example embodiment and with the aid of the figures, only the features required for understanding the invention being illustrated. The following reference numerals are used here: 1: CT system; 2: X-ray tube of the FDSA; 3: detector of the DSA; 4: X-ray tube of the FDSB; 5: detector of the FDSB; 6: gantry housing; 7: patient; 8: displaceable patient couch; 9: system axis; 10: control and computation unit; Prg₁-Prgₙ: computer system 11: beam of the smaller focus/detector system; 12: measuring range of the smaller focus/detector system; 13: beam of the larger focus/detector system; 14: measuring range of the larger focus/detector system; 15: phantom; FDSA: focus/detector system A with X-ray tube 2 and detector 3; FDSB: focus/detector system B with X-ray tube 4 and detector 5; F_{A}: focus of the FDSA; F_{B}: focus of the FDSB; β_{A}: fan angle of the FDSA; β_{B}: fan angle of the FDSB; D_{A}: detector of the FDSA; D_{B}: detector of the FDSB; 16: theoretic profile of the absorption of a projection; 17: measured profile of the absorption in the FDSA; 18: measured profile of the absorption in the FDSB; μ: absorption; k: channel.

In detail,

FIG. 1 shows a schematic of a computed tomography system having two focus/detector systems,

FIG. 2 shows a cross section through a CT system having two focus/detector systems during scanning of a phantom,

FIG. 3 shows an analytically calculated absorption profile of a parallel projection,

FIG. 4 shows a measured absorption profile of the projection angle from FIG. 3 with the aid of a first focus/detector system with wide fan, and

FIG. 5 shows a measured absorption profile of a parallel projection with the same projection angle as in FIGS. 3 and 4 with the aid of the second, smaller focus/detector system.

### DETAILED DESCRIPTION OF THE EXAMPLE EMBODIMENTS

The terminology used herein is for the purpose of describing particular embodiments only and is not intended to be limiting of the present invention. As used herein, the singular forms “a”, “an” and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

Referencing the drawings, wherein like reference numerals designate identical or corresponding parts throughout the several views, example embodiments of the present patent application are hereafter described.

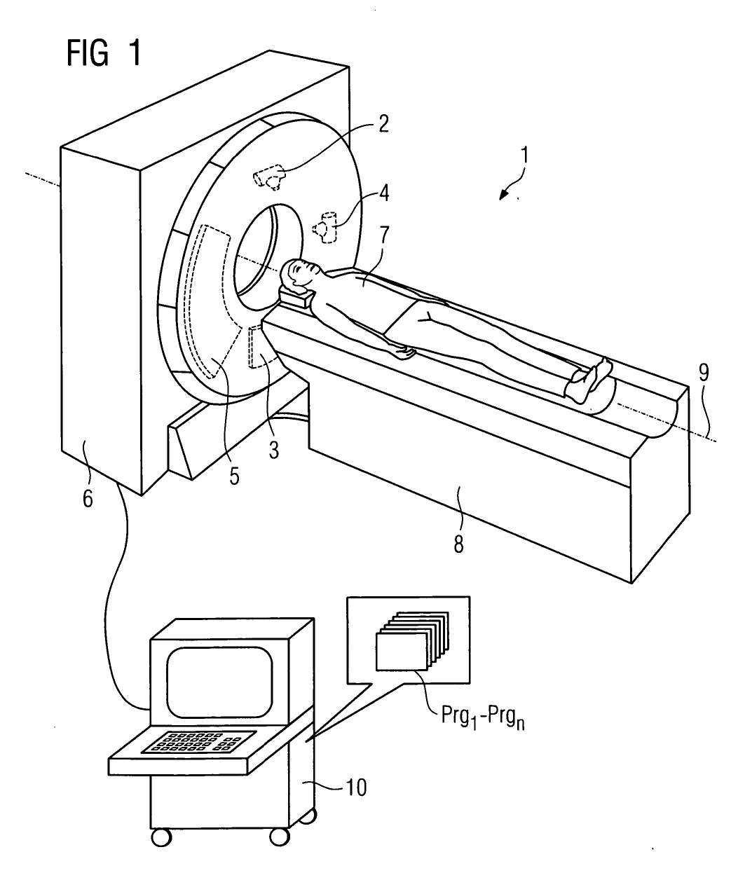

FIG. 1 shows a CT system 1 having a first focus/detector system FDSA with the associated X-ray tube 2 and the oppositely situated detector 3, and a second focus/detector system FDSB with the X-ray tube 4 and the oppositely situated detector 5. The two focus/detector systems are arranged on a gantry (not visible here) that is located in the gantry housing 6. In order to scan the patient, the two focus/detector systems rotate about a system axis 9, while a patient 7 is pushed continuously or in steps through the scanning region of the focus/detector systems with the aid of the displaceable couch 8. The control and evaluation of the measured data takes place by way of a control and computation unit 10 that contains a multiplicity of programs or program modules Prg₁-Prgₙ that can, inter alia, also simulate the method according to an embodiment of the invention for calibrating the system.

It may be pointed out that it is also within the scope of the invention when individual method steps or a number thereof and programs are executed on other computer systems.

It is typical in the case of such CT systems having a number of focus/detector arrangements to mix the measured values of the two focus/detector arrangements and use them to reconstruct CT images or volume data. It has emerged here that there is a clear need to coordinate the individual focus/detector systems with one another so that no artifacts are produced in the reconstruction. It is not sufficient in this case to calibrate each individual focus/detector system for itself—rather, there is also a need to compensate the mutual influences of the individual focus/detector systems so as to prevent the formation of artifacts in the reconstruction of CT images or CT volume data.

According to an embodiment of the invention, calibration matrices are prepared to this end for each focus/detector system, and these not only calibrate the focus/detector systems to another, but exclude the exertion of mutual influence, for example by scattered radiation, by virtue of the fact that a calibration is undertaken on ideal data, that is to say analytically determined data, or that a calibration is carried out on measured data that takes place without the influence of another focus/detector system that is operating simultaneously.

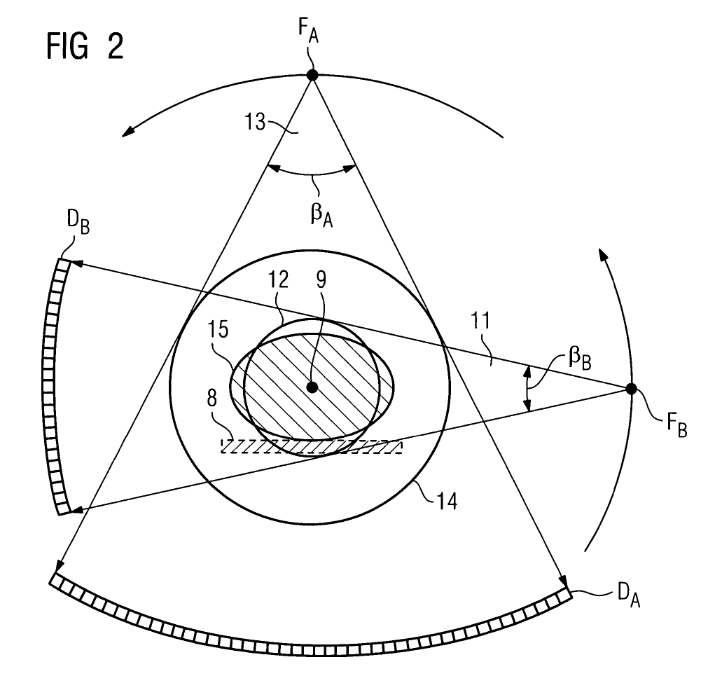

For example, for the calibration according to an embodiment of the invention a phantom 15 such as is shown in FIG. 2 can have its absorption data calculated analytically. In the process, a multiplicity of parallel projections can, for example, be calculated theoretically and compared with the actually measured parallel projections of the two focus/detector systems. Such an arrangement of a phantom 15 in a CT system having two focus/detector systems FDSA and FDSB is illustrated in cross section in FIG. 2. The phantom 15 is arranged on the displaceable couch 8 and is scanned by two focus/detector systems FDSA and FDSB. Here, the focus/detector system FDSA has a focus F_{A} that is situated opposite a detector D_{A}. The focus F_{A} generates a ray fan 13 with a wide fan angle β_{A} that scans a large circular measuring field 14 at the center of rotation owing to the rotation about the system axis 9.

Arranged perpendicular to the first focus/detector system is the second focus/detector system FDSB, which has a focus F_{B} and an oppositely situated detector D_{B}. In accordance with the lesser width of the detector D_{B}, the ray fan 11 emanating from the focus F_{B} and having the fan angle β_{B} is also substantially narrower and scans a smaller measuring field 12 when rotating about the system axis 9.

Situated in the region of the measuring fields 14 and 12 is a phantom 15 that has an approximately elliptical cross section and thereby corresponds approximately to the cross section of a scanned patient. This phantom is generally filled with a substance resembling tissue, preferably water, and it is thereby possible to simulate the mutual influence between the two focus/detector systems FDSA and FDSB.

According to an embodiment of the invention, the theoretical absorption of each ray of the ray fans 13 and 11, if appropriate in a parallel projection, can now be calculated and these theoretical results can be compared with the measured values actually found for the two focus/detector systems during simultaneous operation. It is possible in this way to prepare calibration matrices for the two focus/detector systems which normalize the measured values obtained to the theoretically calculated absorption data such that the two focus/detector systems not only are fitted to one another, but are normalized to the ideal measured values.

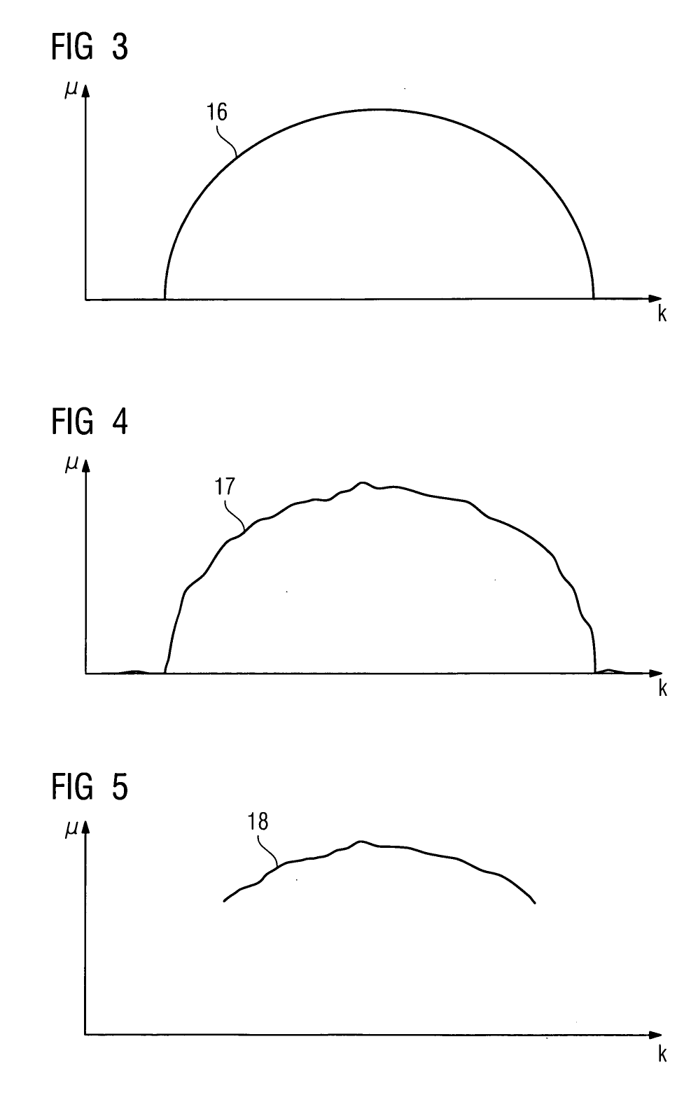

FIGS. 3 to 5 show such a procedure for in each case a single parallel projection at a single projection angle. Here, FIG. 3 illustrates the profile 16 of the absorption values in a coordinate system in which the abscissa reproduces the channels k of a projection, and the ordinate forms the absorption values μ.

FIG. 4 shows a schematic of absorption values, for example measured ones, of a parallel projection with the aid of the larger focus/detector system FDSA and the profile 17 of the absorption in a fashion plotted against the channels k, while FIG. 5 shows, in the same direction of projection, the parallel projection of the second focus/detector system FDSB. In accordance with the smaller fan angle β_{B}, the width of the measured channels in FIG. 5 is also smaller, FIGS. 3 to 5 being arranged such that identical channels also reproduce the geometrically identical ray through the scanned phantom. Thus, a required calibration value can be formed in this way for each measuring ray from the difference between the actually measured absorption values and the ideally found absorption values, and a corresponding calibration matrix can be prepared for each focus/detector system.

In addition, these calibration matrices can be prepared for phantoms of different shape and size such that, in accordance with the actually scanned object, use is made in each case of a calibration matrix that is formed by a corresponding phantom of similar size and similar shape. In addition, given desired intermediate sizes the calibration matrix can be computationally adapted with reference to its extent to the actually measured size of the scanned object.

Thus, according to an embodiment of the invention the obtained measurement results of a scan performed with simultaneous operation of a number of focus/detector systems are processed in such a way that for each X-ray beam in each spatial direction a calibration value is sought that has been determined with the aid of a corresponding phantom as similar as possible to the scanned object, and the reconstruction of the CT data from a mixed position of the measured values of all the focus/detector systems used is not carried out until after a corresponding calibration of the individual measured values.

It is self evident that the above-named features of embodiments of the invention can be used not only in the respectively specified combination, but also in other combinations or on their own, without departing from the framework of the invention.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for calibrating a CT system, comprising:
arranging at least two focus/detector systems, arranged angularly offset from one another, on a rotatable gantry;
using, to scan an object, the angularly offset foci with fanned-open X-ray beams to irradiate respectively oppositely situated detectors, with a multiplicity of detector elements arranged like matrices, while the focus/detector systems rotate about the object;
assigning each detector element, of each focus/detector system, an X-ray beam per angle of rotation of the gantry;
coordinating measured values of the at least two focus/detector systems, with one another individually per measured X-ray beam, before the carrying out of a reconstruction of CT data of the object from at least two different focus/detector systems by use of a calibration matrix (K_{k,s,r}^{FDSA},K_{k,s,r}^{FDSA}) per focus/detector system, each calibration matrix (K_{k,s,r}^{FDSA}, K_{k,s,r}^{FDSB}) being determined in such a way to generate a compensation between measured values during simultaneous operation of the at least two focus/detector systems and absorption data mutually uninfluenced by the number of focus/detector systems.

2. The method as claimed in claim 1, wherein
in at least one angular position of the gantry, a scan of at least one phantom is carried out simultaneously with the aid of all the focus/detector systems,
the attenuation of the X-ray beam at this at least one phantom is calculated for each measured X-ray beam of each focus/detector system, and
each calibration matrix (K_{k,s,r}^{FDSA}, K_{k,s,r}^{FDSB}) is prepared on the basis of the calculated beams, each measured X-ray beam of each focus/detector system being normalized to the calculated attenuation of the corresponding X-ray beam.

3. The method as claimed in claim 2, wherein the calculation of the attenuation values and the scanning of the phantom take place at a single angle of rotation in the case of a rotationally symmetrical phantom, and each calibration matrix (K_{k,s,r}^{FDSA}, K_{k,s,r}^{FDSB}) is prepared independently of the angle of rotation of the gantry.

4. The method as claimed in claim 2, wherein the calculation of the attenuation values and the scanning of the phantom take place for a multiplicity of angles of rotation, and each calibration matrix K_{k,s,r}^{FDSA} , K_{k,s,r}^{FDSB}) is prepared for all the spatial directions of the beams.

5. The method as claimed in claim 1, wherein
a scan is carried out simultaneously with the aid of all the focus/detector systems in at least one angular position of the gantry of at least one phantom,
a scan is carried out with the aid of only one focus/detector system, and the attenuation of the X-ray beams at this at least one phantom is determined without the influence of the at least one other focus/detector system and
each calibration matrix K_{k,s,r}^{FDSA} , K_{k,s,r}^{FDSB}) is prepared on the basis of the attenuation values of the beams determined with the aid of only one focus/detector system, each measured X-ray beam of each focus/detector system being normalized to the individually determined attenuation of the corresponding X-ray beam.

6. The method as claimed in claim 5, wherein the determination of the attenuation of the X-ray beams is carried out by a single focus/detector system, and the scan is carried out for a multiplicity of angles of rotation with the aid of all the focus/detector systems, and each calibration matrix (K_{k,s,r}^{FDSA}, K_{k,s,r}^{FDSB}) is prepared for all the spatial directions of the beams.

7. The method as claimed in claim 1, wherein typical body shapes, for which calibration matrices (K_{k,s,r}^{FDSA}, K_{k,s,r}^{FDSB}) are stored in each case, are used as the phantom, calibration matrices (K_{k,s,r}^{FDSA}, K_{k,s,r}^{FDSB}) being used for the most similar shape and dimension in each case in accordance with the scanned object region.

8. The method as claimed in claim 7, wherein at least one of the adaptation and selection of each calibration matrix (K_{k,s,r}^{FDSA}, K_{k,s,r}^{FDSB}) is performed by at least one topogram recorded before scanning.

9. The method as claimed in claim 7, wherein at least one of the adaptation and selection of each calibration matrix (K_{k,s,r}^{FDSA}, K_{k,s,r}^{FDSB}) is performed on the basis of at least two topograms recorded before scanning.

10. The method as claimed in claim 7, wherein at least one of the adaptation and selection of each calibration matrix (K_{k,s,r}^{FDSA}, K_{k,s,r}^{FDSB}) is performed on the basis of topograms recorded in an angularly offset fashion with the aid of each focus/detector system, the relative recording angles in relation to one another corresponding to the angular offset of the focus/detector systems on the gantry.

11. The method as claimed in claim 7, wherein at least one of the adaptation and selection of each calibration matrix (K_{k,s,r}^{FDSA}, K_{k,s,r}^{FDSB}) is performed with the aid of object shadows measured during scanning.

12. The method as claimed in claim 1, wherein the calibration is carried out by projection.

13. The method as claimed in claim 12, wherein the calibration is carried out in parallel projections.

14. The method as claimed in claim 13, wherein, in the case of a two-focus/detector system, the calibration matrix K_{k,s,r}^{FDSA} of the first focus/detector system (FDSA), and the calibration matrix K_{k,s,r}^{FDSB} of the second focus/detector system (FDSB) are calculated as follows:
$${K}_{k , s , r}^{FDSA} = 1 + \frac{{W}_{k , s , r} ( {x}_{0} , {y}_{0} , {α}_{0}^{FDSA} ) - {h}_{k , s , r}^{FDSA}}{{W}_{k , s , r} ( {x}_{0} , {y}_{0} , {α}_{0}^{FDSA} )}$$
$$and$$
$${K}_{k , s , r}^{FDSB} = 1 + \frac{{W}_{k , s , r} ( {x}_{0} , {y}_{0} , {α}_{0}^{FDSB} ) - {h}_{k , s , r}^{FDSB}}{{W}_{k , s , r} ( {x}_{0} , {y}_{0} , {α}_{0}^{FDSB} )}$$
where W_{k,s,r} (x₀, y₀, α₀^{FDSA}) and W_{k,s,r} (x₀, y₀, α₀^{FDSB}) are the projection values as calculated or measured in individual operation, h_{k,s,r}^{FDSA} are the measured data, obtained during a common scan, of the first focus/detector system, and h_{k,s,r}^{FDSA} are the measured data of the second focus/detector system, k determining the channel of a projection, s determining the row of the detector, r determining the projection number, x₀, y₀ determining the position of the phantom and α₀^{FDSA} and α₀^{FDSB} respectively determining the projection angles of the respective focus/detector system.

15. The method as claimed in claim 1, wherein, in the case of at least one of detectors of different size and of the use of ray fans of different size, the values of the smaller detector or ray fan are calibrated to the values of the larger detector or ray fan.

16. The method as claimed in claim 1, wherein the object is moved along a system axis during the rotation of the focus/detector systems.

17. A computed tomography system comprising:
at least two focus/detector systems to scan an object using different ray fans, attenuation of radiation during passage through the object being determinable therefrom; and
a computation unit, including at least one of programs and program modules stored therein, to determine at least one of tomograms and volume data of the spatial attenuation of the object, the at least one of programs and program modules being used, when run on the computation unit, to coordinate measured values of the at least two focus/detector systems, with one another individually per measured X-ray beam, before carrying out of a reconstruction of CT data of the object from at least two different focus/detector systems by use of a calibration matrix (K_{k,s,r}^{FDSA}, K_{k,s,r}^{FDSB}) per focus/detector system, each calibration matrix (K_{k,s,r}^{FDSA}, K_{k,s,r}^{FDSB}) being determined in such a way to generate a compensation between measured values during simultaneous operation of the at least two focus/detector systems and absorption data mutually uninfluenced by the number of focus/detector systems.

18. A computed tomography system comprising:
at least two focus/detector systems to scan an object using different ray fans arranged angularly offset from one another on a rotatable gantry, attenuation of radiation during passage through the object being determinable therefrom, the angularly offset foci with fanned-open X-ray beams being usable to irradiate respectively oppositely situated detectors, with a multiplicity of detector elements arranged like matrices, while the focus/detector systems rotate about the object, each detector element, of each focus/detector system, being assigned an X-ray beam per angle of rotation of the gantry; and
means for coordinating measured values of the at least two focus/detector systems, with one another individually per measured X-ray beam, before the carrying out of a reconstruction of CT data of the object from at least two different focus/detector systems by use of a calibration matrix (K_{k,s,r}^{FDSA}, K_{k,s,r}^{FDSB}) per focus/detector system, each calibration matrix (K_{k,s,r}^{FDSA}, K_{k,s,r}^{FDSB}) being determined in such a way to generate a compensation between measured values during simultaneous operation of the at least two focus/detector systems and absorption data mutually uninfluenced by the number of focus/detector systems.

19. A computer readable medium including program segments for, when executed on a computer device of a computed tomography system, causing the computed tomography system to implement the method of claim 1.
